(19) United States
(12) Patent Application Publication
(10) Pub. No.: US 2024/0366088 A1
(43) Pub. Date: Nov. 7, 2024

(54) A SYSTEM AND A METHOD FOR DETERMINING COGNITIVE CAPABILITIES BASED ON BRAIN IMAGE

(71) Applicant: Inteneural Networks Inc., Chicago, IL (US)

(72) Inventors: Kris B. SIEMIONOW, Chicago, IL (US); Paul LEWICKI, Tulsa, OK (US)

(21) Appl. No.: 18/574,365

(22) PCT Filed: Jun. 27, 2022
(86) PCT No.: PCT/US2022/035054

§ 371 Date: Dec. 27, 2023

Related U.S. Application Data
(60) Provisional application No. 63/215,490, filed on Jun. 27, 2021.

(51) Int. Cl. A61B 5/00; A61B 5/055; G01R 33/48; G01R 33/56

(52) U.S. Cl. CPC A61B 5/0042; A61B 5/055; G01R 33/4806; G01R 33/5608

(57) ABSTRACT

A method for determining cognitive capabilities of a person includes: receiving a brain Magnetic Resonance Imaging (MRI) volume that represents a brain; segmenting the brain MRI volume into white matter, grey matter and cerebrospinal fluid; selecting white matter and/or grey matter from the segmented volume; determining a convex hull shape; computing the contour of the white matter and/or the grey matter shape and the contour of the convex hull shape; and computing a gyrification index based on a comparison of voxels that constitute the contour of the white matter and/or the grey matter and the contour of the convex hull.

### TECHNICAL FIELD

The present invention relates to determining cognitive capabilities based on brain image.

### BACKGROUND

Searching for a neurobiological understanding of human intellectual capabilities has become possible as a result of analysis of magnetic resonance imaging (MRI) derived data. Brain gyrification, or folding of the cortex, is as highly evolved and variable a characteristic in humans as is intelligence. Gyrification scales with brain size, and a relationship between brain size and intelligence has been demonstrated in humans (although this relation is not straightforward). It is important to note that gyrification shows a large degree of variability that is independent from brain size, suggesting that the former may independently contribute to cognitive abilities and thus supporting a direct investigation of this parameter in the context of intelligence. Moreover, uncovering the regional pattern of such an association could offer insights into evolutionary and neural mechanisms.

Cortical thinning is a widely used and powerful biomarker for measuring disease progression in Alzheimer's disease (AD). Recent research has demonstrated that the differences in cortical folding are progressive and can be detected before formal diagnosis of AD. A number of studies have found localized differences in the appearance and extent of cortical folding between the brains of schizophrenic patients and healthy control subjects. Cortical folding was shown to be decreased bilaterally with age in individuals diagnosed with autism when compared to normal subjects. These findings suggest that the gyrification patterns in autism may be abnormal and could be related to the various cortical anomalies observed in this disorder.

The brain anatomy can be non-invasively visualized using magnetic resonance imaging (MRI). Various types of image contrasts, also called MRI sequences or MRI volumes, can be obtained to identify brain structure or abnormalities. The most common volumes are T1-weighted, T2-weighted, and FLAIR (FLuid Attenuation Inversion Recovery) scans, although there are other methods to derive similar looking imaging using newer image acquisition methods like MAGIC that take advantage of other signal acquisition strategies (such as described in “Synthetic MRI for Clinical Neuroimaging: Results of the Magnetic Resonance Image Compilation (MAGIC) Prospective, Multicenter, Multireader Trial” (by Tanenbaum LN et al., AJNR Am J Neuroradiol. 2017 June;38 (6): 1103-1110). In general, the volumes obtainable by MRI imaging differ by the contrast in which different types of tissue are visualized.

### SUMMARY OF THE INVENTION

There is a need to provide alternative solutions to determining cognitive capabilities based on brain image.

According to the present invention, the cognitive capabilities are determined as a measure of a gyrification index.

In one aspect, the present invention relates to a method for determining cognitive capabilities of a person, the method comprising the following steps: receiving a brain Magnetic Resonance Imaging (MRI) volume that represents a brain; segmenting the brain MRI volume into white matter, grey matter and cerebrospinal fluid; selecting white matter and/or grey matter from the segmented volume; determining a convex hull shape; computing the contour of the white matter and/or the grey matter shape and the contour of the convex hull shape; and computing a gyrification index based on a comparison of voxels that constitute the contour of the white matter and/or the grey matter and the contour of the convex hull.

The method may comprise computing the contours by using a morphological gradient operator.

The method may comprise computing a general gyrification index by dividing the number of nonzero voxels in the contour of the white matter and grey matter shape by the number of nonzero voxels of the contour of the convex hull.

The method may comprise computing a local gyrification index for selected voxels on the contour of the convex hull, by dividing the number of nonzero voxels in the contour of the white matter and grey matter shape by the number of nonzero voxels of the contour of the convex hull within a predetermined neighborhood of the selected voxel.

The neighborhood may comprise a volume having a size of N×N×N voxels, wherein N is from 15 to 21, wherein the selected voxel is positioned in the center of said volume.

The method may comprise computing a region gyrification index by providing a template anatomical region point cloud within which a plurality of regions are defined, registering a local gyrification index point cloud with respect to the template anatomical region point cloud, selecting a region and for each point of the template anatomical region point cloud of that region, finding a closest neighbor in the local gyrification index point cloud, calculating an average of local gyrification index values for all points of that region and outputting the calculated average as the region gyrification index for the selected region.

In another aspect, the invention relates to a computer-implemented system, comprising at least one nontransitory processor-readable storage medium that stores at least one of processor-executable instructions or data; and at least one processor communicably coupled to at least one nontransitory processor readable storage medium, wherein at least one processor is configured to perform the steps of the method according to claim 1.

### BRIEF DESCRIPTION OF DRAWINGS

The present invention is shown by means of exemplary embodiments on a drawing, wherein:

FIG. 1 shows an example of an axial cross-section of an input volume;

FIG. 2 shows a procedure for determining the gyrification index;

FIG. 3A shows a region on the MRI brain image representing an actual brain;

FIG. 3B shows a volume representing only the brain part of the head scan;

FIG. 3C shows two examples of the volume after a skull stripping process;

FIG. 3D shows a segmented volume of FIG. 6C;

FIG. 3E shows a shape after a morphological closing operation;

FIG. 3F shows a contour of the shape from FIG. 6D;

FIG. 3G shows a contour of the shape from FIG. 6E;

FIG. 4A shows a registered local gyrification point cloud;

FIG. 4B shows a color scale for the point cloud of FIG. 4A;

FIG. 4C shows a template anatomical region point cloud;

FIG. 5 shows a method for calculating a region gyrification index;

FIG. 6 shows a computer-implemented system for performing the method described herein.

### DETAILED DESCRIPTION

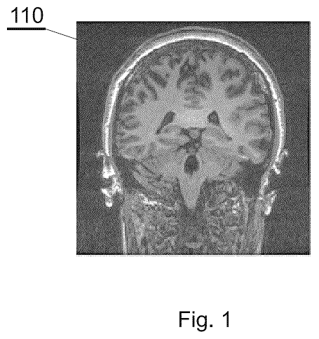

The system as presented herein uses a 3D raw MRI brain image 110 as an input volume, as shown in FIG. 1, such as T1-weighted volume.

The term “volume” as used herein refers to a series of images corresponding to a 3D representation of the brain.

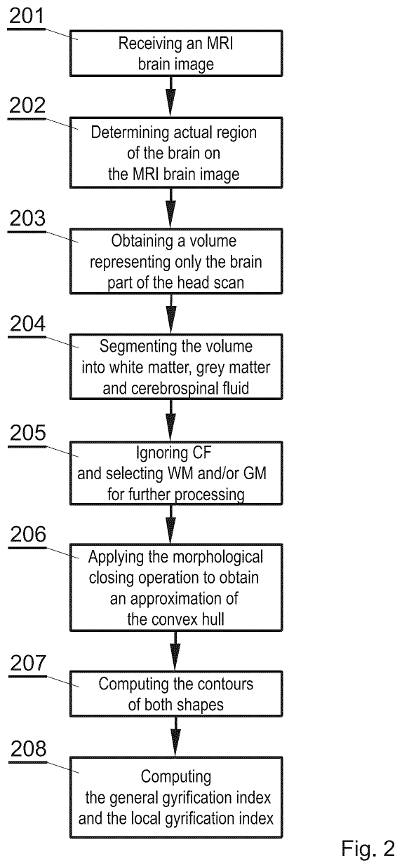

FIG. 2 shows a procedure for determining the gyrification index as a measure of cognitive capabilities of a person.

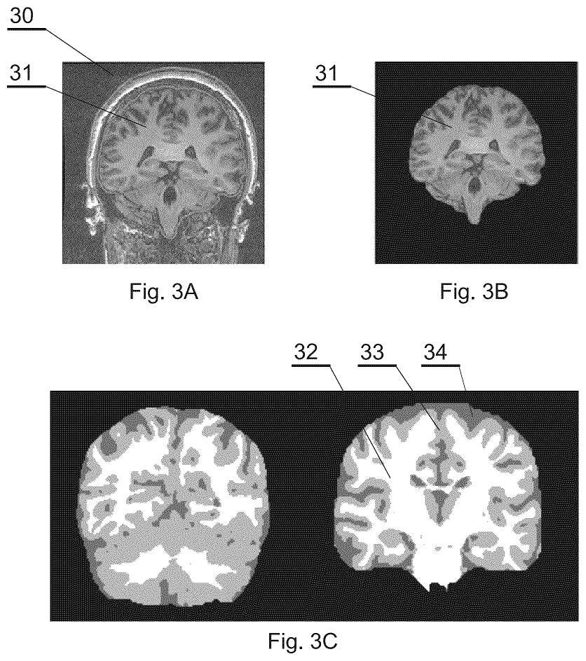

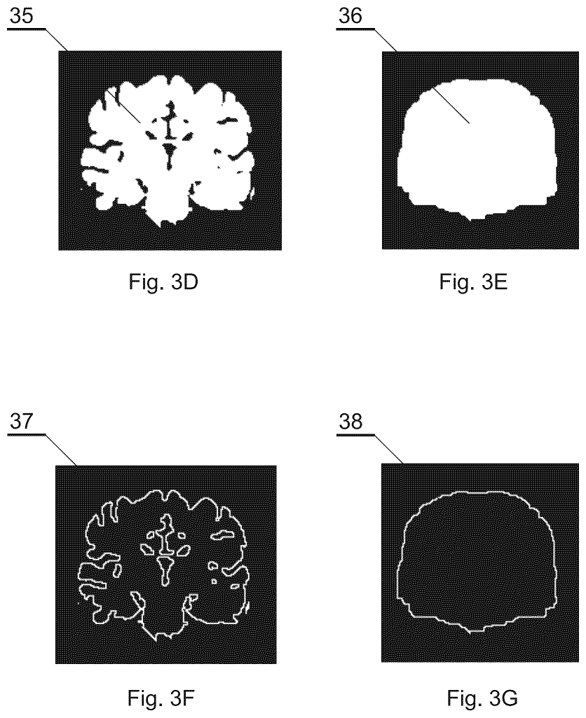

First, at 201, a raw MRI brain image 30 is received as input data, in the form of the input volumes. Next, at 202, a process of skull stripping is started in which a region 31 representing an actual brain is determined on the MRI brain image, such as shown in FIG. 3A. Next, at 203, a volume representing only the brain part 31 of the head scan (FIG. 3B) is obtained, such as t1-weighted image.

Next, at 204, the volume 31 representing only the brain part is segmented into white matter, grey matter and cerebrospinal fluid as presented by two sample results in FIG. 3C wherein white color is the white matter 32, light grey color is the grey matter 33, dark grey color is the cerebrospinal fluid 34.

The steps of skull stripping 202 and segmentation 204 may be performed by means of a convolutional neural network (CNN), such as described in a European patent application EP3470006A1 or any other type of neural network or an algorithm suitable for that purpose. Next, at 205, the cerebrospinal fluid is ignored for further processing and a selection is made whether the gyrification index shall be computed for white matter and/or for grey matter. This can be done by:
computing gyrification of grey matter by treating the cerebrospinal fluid area within the segmented volume as background (and assigning it with a “0” value), and if the gyrification index is to be computed for grey matter, the white and grey matter areas are assigned with a “1” value;
computing gyrification of white matter: the grey matter area is assigned with a “0” value and the white matter area is assigned with a “1” value.

In other words, the white matter and/or the grey matter areas are selected from the brain MRI volume. The result 35 of this operation is presented in FIG. 3D.

Next, at 206, the morphological closing operation is performed in order to ‘close’ the gaps formed by the sulci (i.e., the area within the outer boundary) and get an approximation of the convex hull of the shape 35 from FIG. 3D which results in a shape 36 presented in FIG. 3E. The size of the gap that shall be closed within the shape 35 and is not to be considered as part of the outer boundary can be determined for example by means of a method described by Schaer, Marie, et al. in a publication “A surface-based approach to quantify local cortical gyrification” (IEEE transactions on medical imaging 27.2 (2008): 161-170).

Next, at 207, contours of both shapes 35, 36 (from FIG. 3D and FIG. 3E) are computed using the morphological gradient operator. The morphological gradient operator emphasizes the edges in a 2D or 3D image and indicated the difference between the dilation (local maximum filter) and the erosion (local minimum filter) of the image. The computed contour 37 of the shape 35 from FIG. 3D is presented in FIG. 3F and the computed contour 38 of the shape 36 from FIG. 3E is presented in FIG. 3G.

Finally, at 208, the gyrification index is calculated based on a comparison of voxels that constitute the contour of the white matter or the grey matter and the contour of the convex hull. That gyrification index, which can be either a general gyrification index, a local gyrification index or a region gyrification index, is later provided as a measure of the cognitive capabilities. In order to calculate the general gyrification index, the number of nonzero voxels (i.e., the voxels that aren't black, e.g., they constitute the surface, either the inner or the outer) in the contour 37 of the white matter and grey matter shape is calculated and divided by the number of nonzero voxels of the contour 38 of the convex hull. The more curved the white matter/grey matter shape is, the higher the gyrification index is.

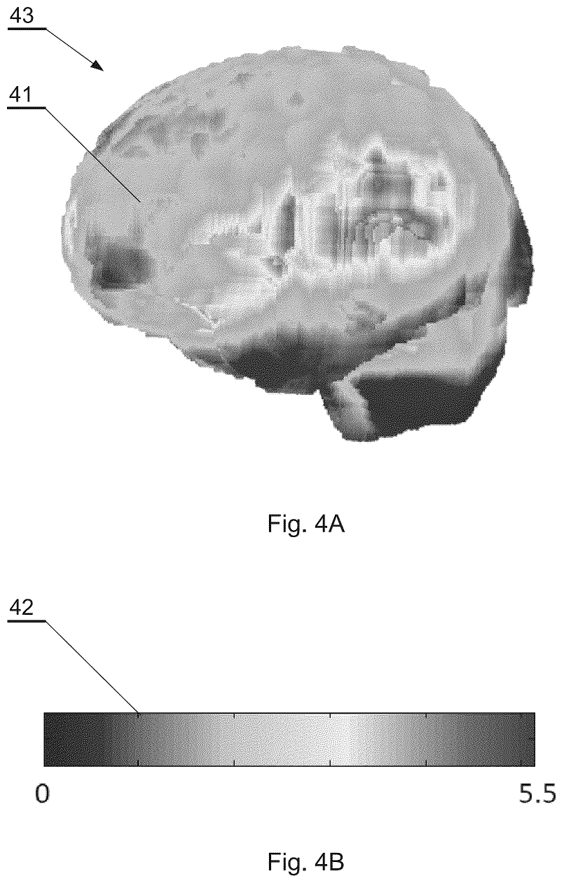

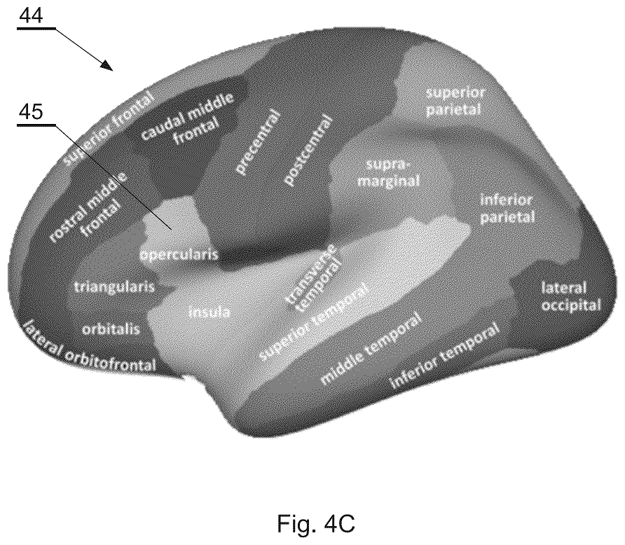

For the local gyrification index computation, the same procedure is performed for selected voxels on the contour 38 of the convex hull, within their N×N×N 3D neighborhood, wherein the particular voxel is positioned in the center of that N×N×N volume. For example, all voxels can be selected to obtain a picture of local gyrification at the whole brain model. N is preferably between 15 and 21 and is an odd natural number. An odd number is preferred for N, because the results are to be localized in a certain voxel and are to be isotropic (to assign the same weight in all directions during the computation). The range 15 to 21 is based on typical dimensions of the brain and corresponds to 15-21 mm size. This way, each voxel 41 of the convex hull has its specific local gyrification index value assigned. That local gyrification index value is calculated in a way equivalent to the general gyrification index but limited to the specified neighborhood—by dividing the number of nonzero voxels in the contour 37 of the white matter and grey matter shape by the number of nonzero voxels of the contour 38 of the convex hull within that specified neighborhood. The Local gyrification index can be represented e.g., as a heatmap (FIG. 4A) wherein a color scale 42 (FIG. 4B) with minimum on 0, maximum on 5.5 is used, though it is just a convention set for this particular case. The color denotes the local gyrification for a given point on the surface of the brain. Therefore, the set of voxels 41 forms a registered local gyrification point cloud 43.

By applying the shell/contour voxel count, which is a reasonably good approximation of the surface area, the computations are performed faster as compared to mesh surface-like representation.

The procedure shown in FIG. 2 allows to determine the local and general gyrification index, to measure the magnitude of cortical convolutions on the surface of the mammalian brain, in a fast and reliable manner. The gyrification index serves as a simple numerical measure that can relate to a patient's sex and age group. Generally, the higher the gyrification index, the better. The global gyrification index is an overall measure, whereas the local gyrification index measure enables to quantify individual regions of the brain. The gyrification index allows to estimate the cognitive capabilities of a person. The present invention provides a measure of actual cortical folding (i.e., how much ‘wrinkles’ the brain has and where). The gyrification value can be compared to gyrification values of other patients in the same age group to see whether it is higher or lower and how much.

FIG. 4C shows a template anatomical region point cloud 44 including a plurality of predefined anatomical regions 45 of a brain.

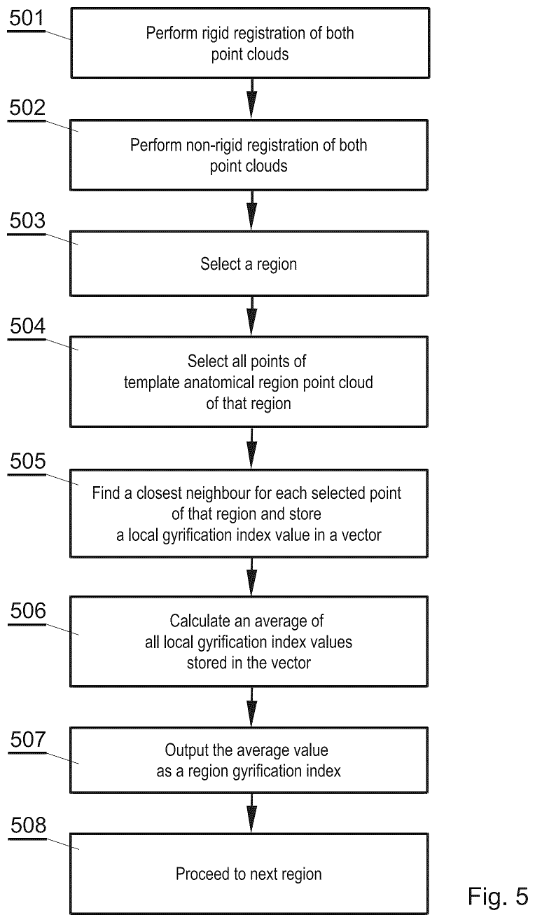

FIG. 5 shows a procedure for determining a region gyrification index to calculate a gyrification index for a particular anatomical region of the brain, which can follow the computation of the local gyrification index of step 208. First, in step 501, a rigid registration is performed, by aligning the registered local gyrification point cloud 43 with the template anatomical region point cloud 44, by means of a rigid point cloud registration algorithm, such as iterative closest point algorithm for the point clouds. Next, in step 502, a non-rigid registration is performed to accurately align the surfaces of the point clouds, so that the registered local gyrification point cloud 43 is aligned with the template anatomical region point cloud 44. Once the point clouds are aligned, the region gyrification index is performed for each region of the template anatomical region point cloud 44. In step 503 a region is selected. Next, in step 504, points of the template anatomical region point cloud 44 are selected that belong to that region. In step 505, for each point of the selected set of points of the template anatomical region point cloud 44, a closest neighbor (for example, in terms of the Euclidean distance) in the registered local gyrification point cloud 43 is selected and a value of the local gyrification index for that nearest neighbor is stored in a vector. In step 506, once all points of the region are processed, and all the values of the local gyrification index are collected, the average value of the values stored in the vector is calculated. The average value is output in step 507 as the region gyrification index. The procedure continues in step 508 to a next region.

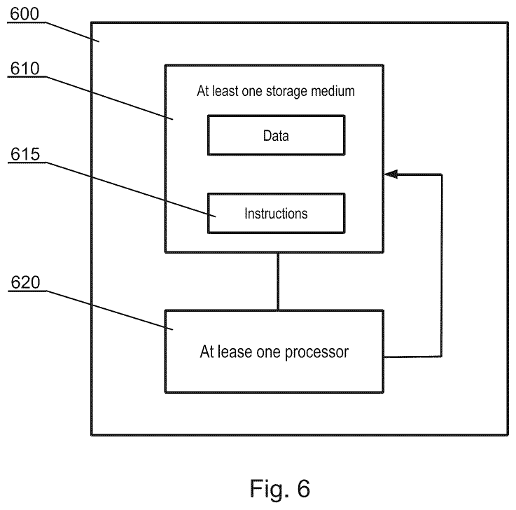

Consequently, all regions of the template anatomical region point cloud 44 are provided with the region gyrification index value, which may have a diagnostic value. The functionality described herein can be implemented in a computer-implemented system 600, such as shown in FIG. 6. The system may include at least one non-transitory processor-readable storage medium that stores at least one of processor-executable instructions or data and at least one processor communicably coupled to at least one non-transitory processor-readable storage medium. At least one processor is configured to perform the steps of the methods presented herein. The computer-implemented system 600, for example a machine-learning system, may include at least one non-transitory processor-readable storage medium 610 that stores at least one of processor-executable instructions 615 or data; and at least one processor 620 communicably coupled to the at least one non-transitory processor-readable storage medium 610. At least one processor 620 may be configured to (by executing the instructions 615) to perform the steps of the method of FIG. 2 or FIG. 5.

Although the invention is presented in the drawings and the description and in relation to its preferred embodiments, these embodiments do not restrict nor limit the presented invention. It is therefore evident that changes, which come within the meaning and range of equivalency of the essence of the invention, may be made. The presented embodiments are therefore to be considered in all aspects as illustrative and not restrictive. According to the abovementioned, the scope of the invention is not restricted to the presented embodiments but is indicated by the appended claims.

1. A method for determining cognitive capabilities of a person, the method comprising the following steps:
receiving a brain Magnetic Resonance Imaging (MRI) volume that represents a brain;
segmenting the brain MRI volume into white matter, grey matter and cerebrospinal fluid;
selecting white matter and/or grey matter from the segmented volume;
determining a convex hull shape;
computing the contour of the white matter and/or the grey matter shape and the contour of the convex hull shape; and
computing a gyrification index based on a comparison of voxels that constitute the contour of the white matter and/or the grey matter and the contour of the convex hull.

2. The method according to claim 1, comprising computing the contours by using a morphological gradient operator.

3. The method according to claim 1, comprising computing a general gyrification index by dividing the number of nonzero voxels in the contour of the white matter and grey matter shape by the number of nonzero voxels of the contour of the convex hull.

4. The method according to claim 1, comprising computing a local gyrification index for selected voxels on the contour of the convex hull, by dividing the number of nonzero voxels in the contour of the white matter and grey matter shape by the number of nonzero voxels of the contour of the convex hull within a predetermined neighborhood of the selected voxel.

5. The method according to claim 4, wherein the neighborhood comprises a volume having a size of N×N×N voxels, wherein N is from 15 to 21, wherein the selected voxel is positioned in the center of said volume.

6. The method according to claim 1, comprising computing a region gyrification index by providing a template anatomical region point cloud within which a plurality of regions are defined, registering a local gyrification index point cloud with respect to the template anatomical region point cloud, selecting a region and for each point of the template anatomical region point cloud of that region, finding a closest neighbor in the local gyrification index point cloud, calculating an average of local gyrification index values for all points of that region and outputting the calculated average as the region gyrification index for the selected region.

7. A computer-implemented system, comprising at least one nontransitory processor-readable storage medium that stores at least one of processor-executable instructions or data; and at least one processor communicably coupled to at least one nontransitory processor readable storage medium, wherein at least one processor is configured to perform the steps of the method according to claim 1.
